(19) United States
(12) Patent Application Publication
(10) Pub. No.: US 2008/0064995 A1
(43) Pub. Date: Mar. 13, 2008

(54) Elastic compressor for a pelvic band

(76) Inventor: Pang-Ching Chiang, Taipei (TW)

Correspondence Address: COOPER & DUNHAM, LLP, 1185 AVENUE OF THE AMERICAS, NEW YORK, NY, 10036, US

(21) Appl. No.: 11/519,529

(22) Filed: Sep. 12, 2006

(51) Int. Cl. A61F 5/00

(52) U.S. Cl. 602/5

(57) ABSTRACT

An elastic compressor for a pelvic band has an inner belt and an outer belt. The inner belt has a first end, a second end, an inner surface and two male fasteners. The male fasteners are mounted on the inner surface and are located adjacent respectively to the first end and the second end. The outer belt is attached to the inner belt and has a first end, a second end, an inner surface and a male fastener. The male fastener is mounted on the inner surface adjacent to the first end. After a user wear a pelvic band on the pelvis, the user can attach the male fastener of the inner belt and the male fastener of the outer belt to the pelvic band.

### BACKGROUND OF THE INVENTION

1. Field of the Invention

The present invention relates to a device for stability of the pelvis, and especially an elastic compressor for a pelvic band for holding the pelvis in stable.

2. Description of the Related Art

When a force pulls the pelvis such as in an accident or expectant mothers, the pelvis will be injured. Therefore, a conventional device for stability of the pelvis is worn on the pelvis to position the pelvis and increase the mend of the pelvis.

The device for stability of the pelvis comprises a pelvic band and two compressors. The pelvic band comprises two ends, an outer surface, an inner surface, a female fastener and a male fastener. The female fastener is mounted on the outer surface. The male fastener is mounted on the inner surface adjacent to one of the ends. The compressors are mounted on the pelvic band and each compressor comprises a male fastener, an inner surface and a free end. The male fastener is mounted on the inner surface adjacent to the free end. When wearing the pelvic band on the pelvis, a user can attach the male fastener of the compressors to the female fastener of the pelvic band to increase the pressure. However, the compressor is mounted securely on the pelvic band and cannot be moved to a desired place on the pelvic band.

With reference to FIGS. 5 and 6, another conventional device for stability of the pelvis comprises a pelvic band (50) and two compressors (60). The pelvic band (50) comprises two ends, an outer surface, an inner surface, a female fastener and a male fastener. The female fastener is mounted on the outer surface. The male fastener is mounted on the inner surface adjacent to one of the ends and is attached detachably to the female fastener. The compressors (60) are attached detachably to the female fastener of the pelvic band (50) and each compressor (60) comprises an inner surface, two ends, two male fasteners (61,62). The male fasteners (61,62) are mounted on the inner surface adjacent to the ends of the compressor (60). When wearing the pelvic band on the pelvis, the user can attach one of the male fasteners (61) of each compressor (60) to the female fastener of the pelvic band (50). Then the user pulls the other male fastener (62) of the compressors (60) away from the male fastener (61) to increase the pressure. However, the range and the force provided by the compressors (60) are limited.

### SUMMARY OF THE INVENTION

The objective of the present invention is to provide an elastic compressor for a pelvic band to be more stability of the pelvis.

To achieve the foregoing objective, an elastic compressor for a pelvic band in accordance with the present invention has an inner belt and an outer belt. The inner belt has a first end, a second end, an inner surface and two male fasteners. The male fasteners are mounted on the inner surface and are located adjacent respectively to the first end and the second end. The outer belt is attached to the inner belt and has a first end, a second end, an inner surface and a male fastener. The male fastener is mounted on the inner surface adjacent to the first end. After a user wear a pelvic band on the pelvis, the user can attach the male fasteners of the inner belt and the male fastener of the outer belt to the pelvic band.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of an elastic compressor for a pelvic band in accordance with the present invention;

FIG. 2 is a perspective view of the elastic compressor in FIG. 1 with the inner belt attached to an outer belt;

FIG. 3 is an operational perspective view of the elastic compressor in FIG. 1 with the pelvic band worn on the pelvis of a user;

FIG. 4 is a top view in partial section of the elastic compressor in FIG. 3;

FIG. 5 is a top view in partial section of a conventional pelvic band and a conventional elastic compressor in accordance with the prior art; and

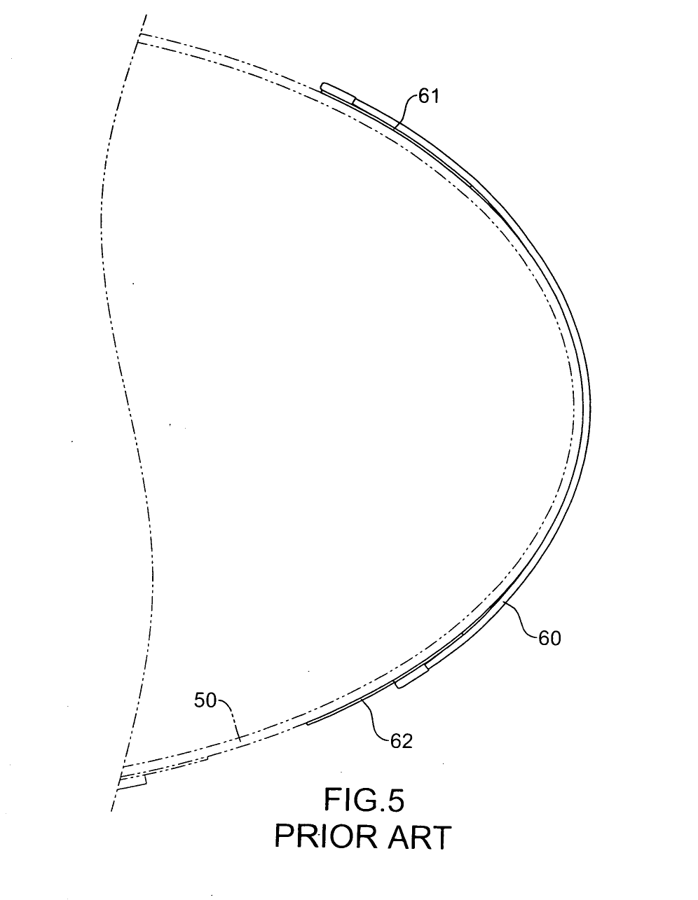

FIG. 6 is a perspective view of the conventional elastic compressor in accordance with the prior art.

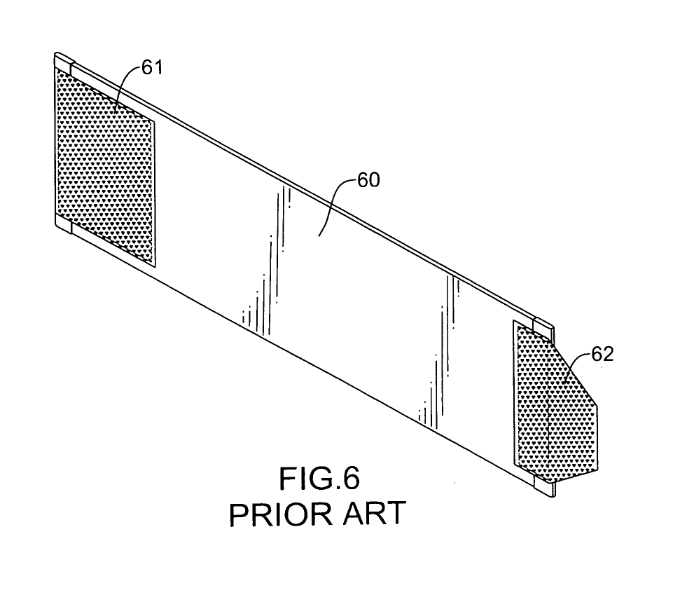

### DETAILED DESCRIPTION OF THE PREFERRED EMBODIMENTS

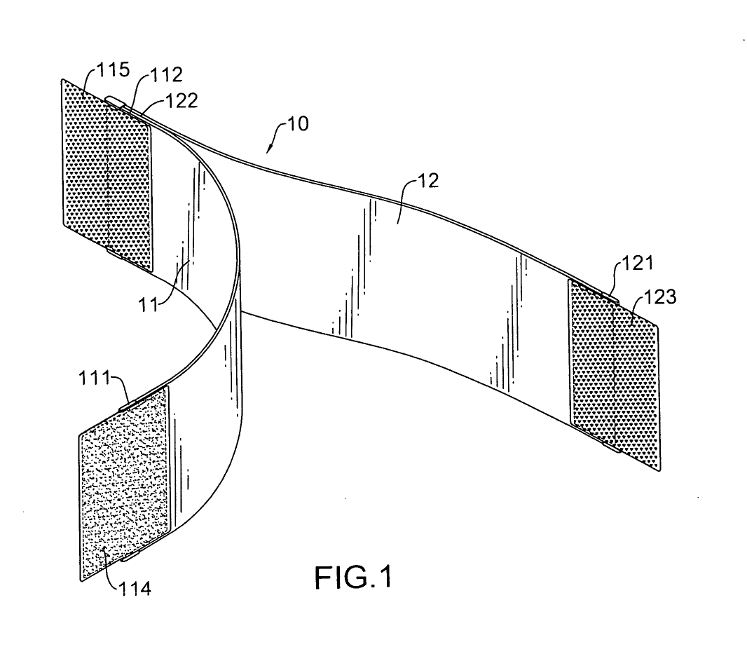

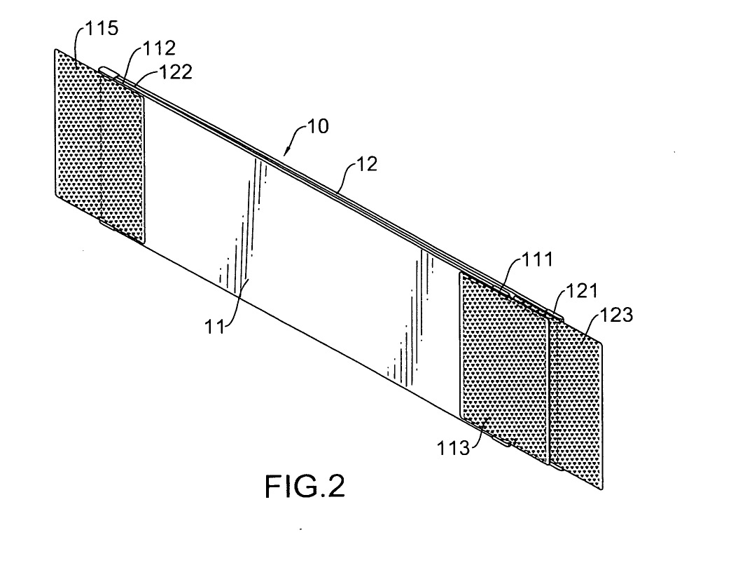

With reference to FIGS. 1 and 2, an elastic compressor for a pelvic band in accordance with the present invention comprises an inner belt (11) and an outer belt (12).

The inner belt (11) comprises a first end (111), a second end (112), an inner surface, an outer surface, two male fasteners (113, 115) and a female fastener (114). The male fasteners (113, 115) are mounted on the inner surface and are located respectively adjacent to the first end (111) and the second end (112). The female fastener (114) is mounted on the outer surface adjacent to the first end (111).

The outer belt (12) is attached to the inner belt (11), may be longer than the inner belt (11) and comprises a first end (121), a second end (122), an inner surface and a male fastener (123). The second end (122) of the outer belt (12) is attached securely to the second end (112) of the inner belt (11) and may be sewed on the second end (112) of the inner belt (11). The male fastener (123) is mounted on the inner surface adjacent to the first end (121).

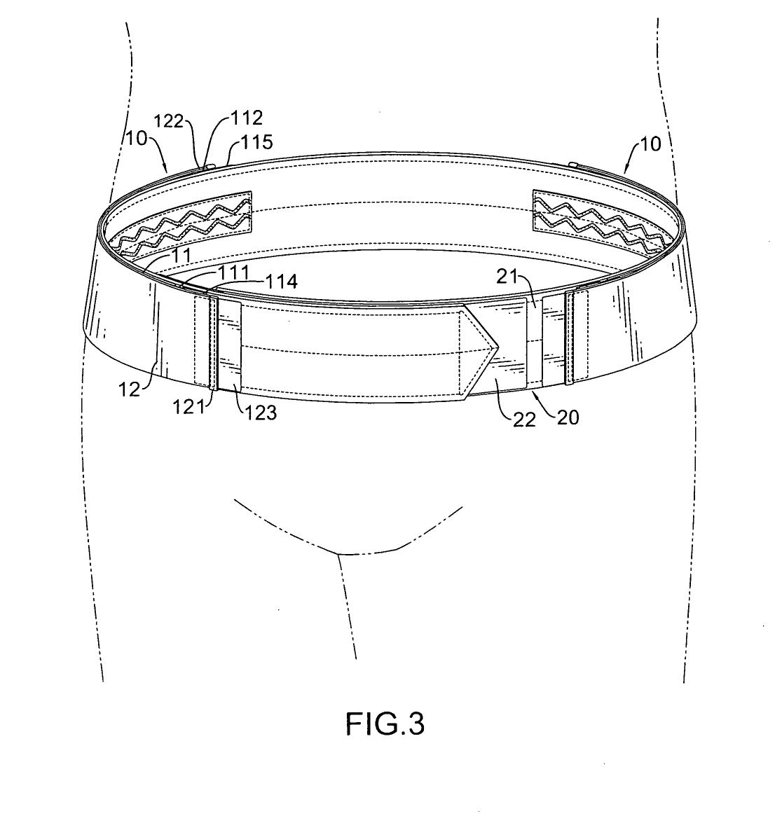

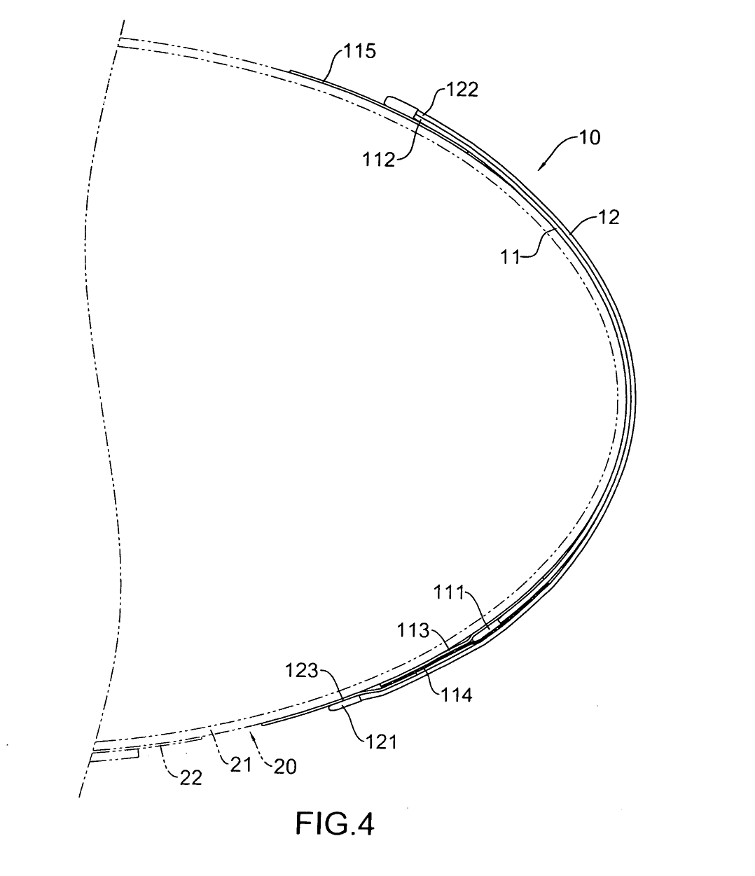

With reference to FIGS. 3 and 4, the elastic compressor (10) attaches to a pelvic band (20). The pelvic band (20) comprises an outer surface, an inner surface, two ends, a female fastener (21) and a male fastener (22). The female fastener (21) is mounted on the outer surface. The male fastener (22) is mounted on the inner surface adjacent to one of the ends and is attached detachably to the female fastener (21).

When wearing the pelvic band (20), a user attaches the male fastener (22) to the female fastener (21) so the pelvic band (20) can steady the pelvic. Then the user attaches the male fastener (115) of the inner belt (11) to the female fastener (21) of the pelvic band (20) and pulls the first end (111) of the inner belt (11) in an opposing direction from the second end (112) to attach the male fastener (113) to the female fastener (21) of the pelvic band (20). When the user feel enough pressure, the user can attach the male fastener (123) of the outer belt (12) to the female fastener (114) of the inner belt (11). When the user needs more pressure, the user can pull the first end (121) of the outer belt (12) more away from the second end (122) so the male fastener (123) can be attached to the female fastener (21) of the pelvic band (20).

The outer belt (12) is sewed stably on the inner belt (11). Therefore, the user can attach the outer belt (12) of the elastic compressor to the pelvic band (10) to enhance pressure so the elastic compressor can stably support the pelvis and increase the rate of the mend of the pelvis.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An elastic compressor for a pelvic band comprising
an inner belt having
a first end;
a second end;
an inner surface;
an outer surface;
two male fasteners mounted on the inner surface and located adjacent respectively to the first end and the second end; and
a female fastener mounted on the outer surface adjacent to the first end; and
an outer belt attached to the inner belt and having
a first end;
a second end attached securely to the second end of the inner belt;
an inner surface; and
a male fastener mounted on the inner surface adjacent to the first end of the outer belt.

2. The elastic compressor for a pelvic band as claimed in claim 1, wherein the second end of the outer belt is sewed on the second end of the inner belt.

3. The elastic compressor for a pelvic band as claimed in claim 1, wherein the outer belt is longer than the inner belt.
